(19) United States
(12) Patent Application Publication
(10) Pub. No.: US 2017/0028179 A1
(43) Pub. Date: Feb. 2, 2017

(54) SUSTAINED DRUG-RELEASING ARTIFICIAL DENTAL CROWN

(71) Applicant: 3D GLOBAL BIOTECH INC., Taipei City (TW)

(72) Inventor: Keng-Liang OU, Kaohsiung City (TW)

(73) Assignee: 3D GLOBAL BIOTECH INC., Taipei City (TW)

(21) Appl. No.: 14/946,060

(22) Filed: Nov. 19, 2015

(30) Foreign Application Priority Data
Jul. 29, 2015 (TW) 104124608

(51) Int. Cl. A61M 31/00; A61C 5/08

(52) U.S. Cl. CPC A61M 31/002; A61C 5/08

(57) ABSTRACT

Provided is a sustained drug-releasing artificial dental crown having a drug container, a drug-supplying microfluidic channel, and a drug-releasing microfluidic channel in the crown body. Medications can be added into the drug container through the drug-supplying microfluidic channel. The medications housed in the drug container can be released to the dental caries slowly and continuously over an extended period through the drug-releasing microfluidic channel. The sustained drug-releasing artificial dental crown provides a crown body to cap the dental caries or dental implant post to treat the dental disease or to wash the dental implant post for a prolonged time. In addition, the medications can be locally deposited on the affected areas by the sustained drug-releasing artificial dental crown. The sustained drug-releasing artificial dental crown can help to reduce the dosage of medications to attain the estimated efficacy and to prevent the side effects by oral administration.

### BACKGROUND OF THE INVENTION

1. Field of the Invention

The present invention relates to an artificial dental crown, and more particularly to a sustained drug-releasing artificial dental crown that has a drug container to house the medications and the medications can be released to dental caries slowly over an extended period.

2. Description of the Related Art

Currently the periodontal disease can be treated by scaling and root planing, periodontal debridment, periodontal surgery, and guided tissue regeneration. After the above therapies, patients also need to take care of the dental hygiene by themselves. Once the oral hygiene is not maintained properly, the inflammation of the nerves or the gingival will be induced. The dentist may consider administration of the antibiotics to treat the infection or inflammation.

There are two kinds of methods to administrate the antibiotics including the oral administration and the local deposition. Both of the methods have defects. Antibiotics by oral administration will be systemically absorbed by the body and then affect the function of the liver and the kidney, especially for pregnant females. The antibiotics may inhibit the growth of the fetus and harm the health of the fetus. In some cases patients need to take the antibiotics many times a day. The efficacy of the antibiotics is hard to attain if patients forget to take the antibiotics on time. Furthermore, taking the antibiotics by local depositing is also hard to attain the estimated efficacy because the antibiotics are diluted by the saliva in patients' mouths. Patients cannot have any food intake over a period of time after local deposition of the medications, causing quite an inconvenience in daily life.

On the other hand, a tooth crowned with a conventional dental crown cannot be locally deposited with the medications because the tooth is encased by the conventional dental crown. As such, the patients can only take the medications by oral administration. Defects about taking the antibiotics by oral administration are as described above. Administration methods for medications for oral diseases that would not harm patients' health are needed.

### SUMMARY OF THE INVENTION

An objective of the present invention is to provide a sustained drug-releasing artificial dental crown for the dental caries. The sustained drug-releasing artificial dental crown can house the medications and release the medications to the dental caries slowly and continuously over an extended period by local deposition. In addition, the sustained drug-releasing artificial dental crown can reduce the consumed dosage of medications to the minimum to attain the estimated efficacy. The sustained drug-releasing artificial dental crown can help to treat the oral diseases without requiring the patients to take medications by oral administration. It is safe and convenient to dental patients.

To achieve the foregoing objective, the sustained drug-releasing artificial dental crown comprises:

a crown body comprising an upper portion, a bottom portion, a sidewall connected to the upper portion, and a cavity encompassed by the sidewall below the upper portion;

a drug container defined inside the upper portion of the crown body;

a drug-supplying microfluidic channel connecting the drug container and an exterior of the crown body; and

a drug-releasing microfluidic channel connecting the drug container and the cavity.

The advantage of the present invention is that the sustained drug-releasing artificial dental crown has a drug container, a drug-supplying microfluidic channel, and a drug-releasing microfluidic channel in the crown body. The above structures can house the medications and release the medications to affected areas slowly and continuously over an extended period by local and centralized deposition. The sustained drug-releasing artificial dental crown also helps to decrease the dosage of the medications and prevents the medications from delivering into the other organs in the patients' body. The present invention reduces the side effects of medications significantly and it is convenient in administration for a long-term therapy.

Particularly, the sidewall is gradually thinner from the upper portion towards the bottom portion and the drug container is extended inside the sidewall from the upper portion of the crown body. The advantage of the present invention provides an enlarged drug container to house more medications in the crown body. This arrangement can extend the period of the drug release. Furthermore, the bigger drug container can also house more detergents, disinfectants, or antibacterials to maintain the oral hygiene around the crown body for a prolonged time.

More particularly, the sustained drug-releasing artificial dental crown has multiple drug-releasing microfluidic channels. The advantage of the present invention is that the medications in the drug container can be delivered to affected areas through multiple drug-releasing microfluidic channels more evenly. If one of the microfluidic channels is blocked, the other microfluidic channels can release the medications continuously.

### BRIEF DESCRIPTION OF THE DRAWINGS

FIG. 1 is a perspective view of the sustained drug-releasing artificial dental crown in accordance with the present invention;

FIG. 2 is a cross-sectional view of the sustained drug-releasing artificial dental crown in accordance with the present invention;

FIG. 3 is an operational view of the sustained drug-releasing artificial dental crown in accordance with the present invention, shown in use;

FIG. 4 is a schematic view of the sustained drug-releasing artificial dental crown in accordance with the present invention, shown capping a tooth;

FIG. 5 is a schematic view of the sustained drug-releasing artificial dental crown in accordance with the present invention, shown capping a dental implant post; and

FIG. 6 is a cross-sectional view of another embodiment of the sustained drug-releasing artificial dental crown in accordance with the present invention.

### DETAILED DESCRIPTION OF THE INVENTION

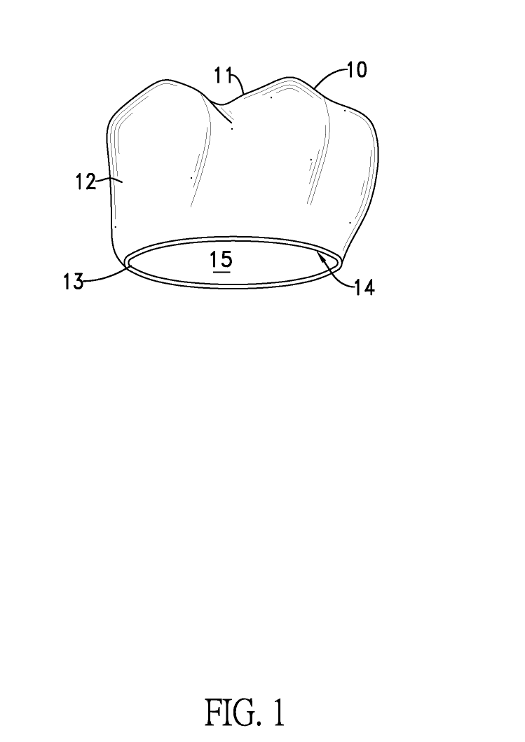

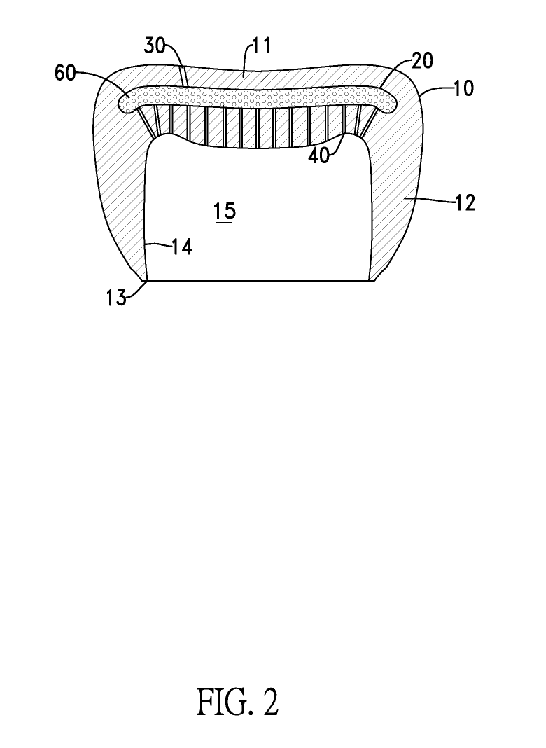

With reference to FIGS. 1 and 2, a sustained drug-releasing artificial dental crown in accordance with the present invention comprises a crown body 10, a drug container 20, a drug-supplying microfluidic channel 30, and multiple drug-releasing microfluidic channels 40.

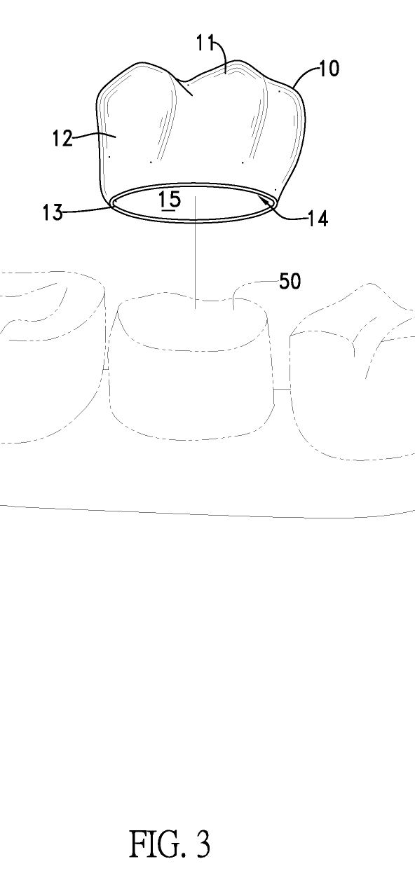

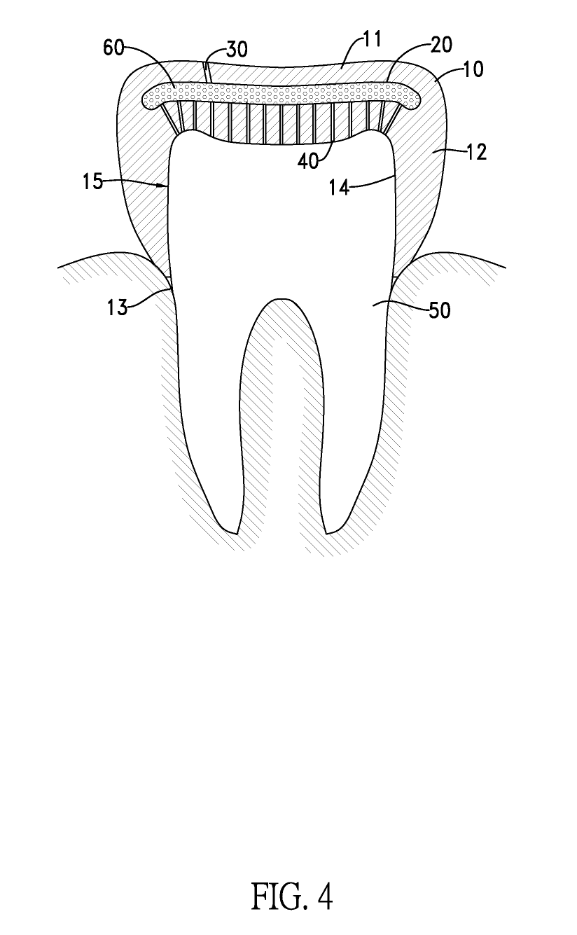

The crown body 10 includes an upper portion 11, a sidewall 12, and a bottom portion 13. The sidewall 12 is connected between the upper portion 11 and the bottom portion 13. The upper portion 11, the sidewall 12, and the bottom portion 13 are integrated. The sidewall 12 is gradually thinner from the upper portion 11 towards the bottom portion 13. The crown body 10 further comprises a cavity 14, and the cavity 14 is encompassed by the sidewall 12 below the upper portion 11. With reference to FIGS. 3 and 4, the cavity 14 is used as a receiving space 15 for a tooth 50 to treat oral diseases.

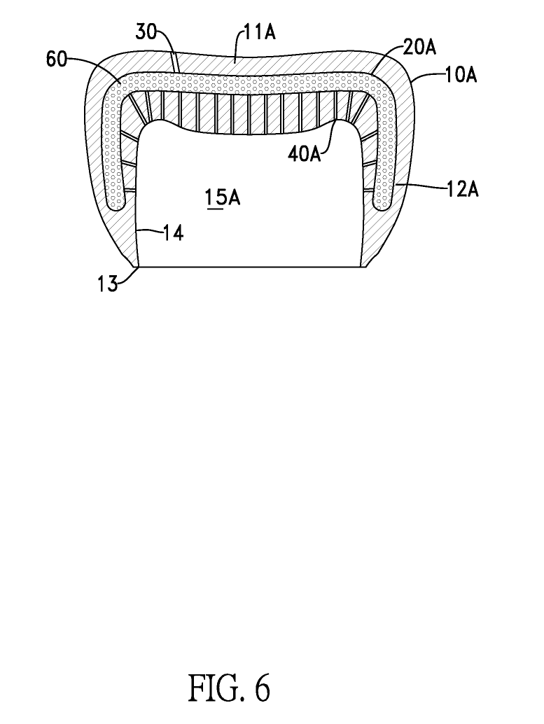

The drug container 20 is primarily built in the upper portion 11 of the crown body 10. Furthermore, the drug container 20 also can extend inside the sidewall 12 from the upper portion 11 of the crown body 10 as shown in FIG. 6. Any kind of medications can be housed in the drug container 20 to treat oral diseases.

The drug-supplying microfluidic channel 30 is formed in the upper portion 11 of the crown body 10. The drug-supplying microfluidic channel 30 connects the drug container 20 and an exterior of the crown body 10. Medications for treating the oral diseases can be added into the drug container 20 through the drug-supplying microfluidic channel 30.

The multiple drug-releasing microfluidic channels 40 are formed in the upper portion 11 of the crown body 10 and the sidewall 12 of the crown body 10. The multiple drug-releasing microfluidic channels 40 connect the drug container 20 and the receiving space 15. The medications housed in the drug container 20 can be released slowly and continuously over an extended period to the tooth 50 encased in the receiving space 15 by the drug-releasing microfluidic channels 40. The purpose of the sustained release therapy for the dental caries or the oral diseases can be achieved by the present invention.

The sustained drug-releasing artificial dental crown can be fabricated by a 3D printing machine or an additive manufacturing machine. The sustained drug-releasing artificial dental crown can be made of medical polymer materials such as Polymethylmethacrylate (PMMA), Polyethylene (PE), Silica gel, Polyeater, Polytetrafluoroethene (PTFE), and Polyurethane (PU). The sustained drug-releasing artificial dental crown can be constructed and custom-made according to shapes and sizes of the teeth 50 for different patients. The 3D printing technology overcomes complex structures and difficult manufacturing processes of the sustained drug-releasing artificial dental crown. Each decayed tooth 50 has an appropriate and exclusive dental crown. The tooth 50 can be encased by the sustained drug-releasing artificial dental crown closely. The medications would hardly overflow through the intervals between the tooth 50 and the sustained drug-releasing artificial dental crown. The optimal effects of the therapy can be achieved from the medications by the present invention.

The coping can be made of medical metal materials such as stainless-steel, titanium, alloy of titanium, and alloy of nickel and chromium. The all ceramic crown can be made of medical ceramics materials such as aluminium oxide, zirconium dioxide, and glass-ceramic. The coping and the all ceramic crown can be formed by a sintering process at high temperature. The above materials do not induce the host immune response and the transplant rejection when the materials contact the human tissue, body fluid or blood. These materials are safe to use for dentist equipments and clinical therapies.

FIG. 4 shows an operational view of the sustained drug-releasing artificial dental crown capping a tooth 50. The cavity 14 fits to the top of the tooth 50, and the crown body 10 encases the top of the tooth 50 closely. The medications 60 can be added into the drug container 20 through the drug-supplying microfluidic channel 30, and then the medications 60 can be released slowly and continuously to the surface of the tooth 50 through the drug-releasing microfluidic channels 40. The medications 60 are locally deposited in the tooth 50 through the sustained drug-releasing artificial dental crown of the present invention. The present invention is effective to prevent the dilution of the medications 60 by the saliva in the mouth, and thereby the present invention also can avoid reducing the therapeutic efficacy of the medications 60.

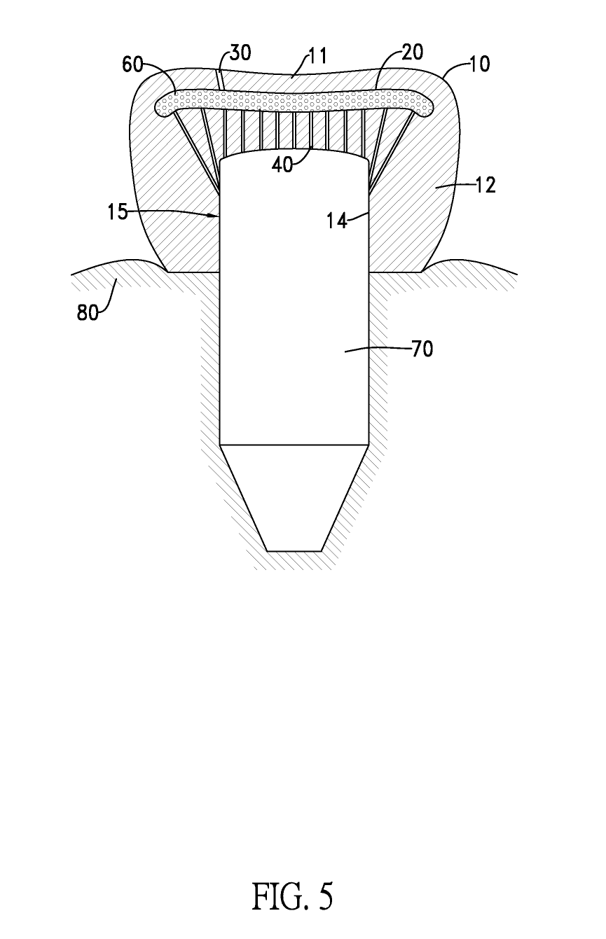

With reference to FIG. 5, the sustained drug-releasing artificial dental crown also can be capped on a dental implant post 70. After removing a tooth from the dental alveolus, the dentist implants a dental implant post 70 into the dental alveolus at the same site. The top of the dental implant post 70 protrudes out of the gingival, and the top of the dental implant post 70 is capped by the sustained drug-releasing artificial dental crown. The cavity 14 of the sustained drug-releasing artificial dental crown fits to the top of the dental implant post 70, and the crown body 10 encases the top of the dental implant post 70 closely. In this situation, the sustained drug-releasing artificial dental crown is used as a denture.

The detergent, the disinfectant or the antibacterial can be added into the drug container 20 through the drug-supplying microfluidic channel 30, and then the detergent, the disinfectant or the antibacterial can be slowly and continuously released to the surface of the dental implant post 70 through the drug-releasing microfluidic channels 40. The present invention is effective to prevent the gingivitis or other oral diseases to the denture.

Furthermore, the drug-supplying microfluidic channel 30 can be sealed by the polymer or the resin when the medications 60 or the disinfectant fully fills the drug container 20. This can prevent the medications 60 or the disinfectant from overflowing into the mouth and then into the stomach along the esophagus. As the medications 60 or the disinfectant may be hazardous to the health, this also can avoid reducing the efficacy of the medications 60 or the disinfectant if the medications 60 or the disinfectant overflows into the mouth.

In addition, the velocity of the sustained release of medications can be controlled by the number of the drug-releasing microfluidic channels 40 and the diameters of the drug-releasing microfluidic channels 40.

With reference to FIG. 6, another embodiment of the sustained drug-releasing artificial dental crown is that the drug container 20A extends inside the upper portion 11A and the sidewall 12A. The drug container 20A is enlarged and can house more medications. In addition, the number of the drug-releasing microfluidic channels 40A increases between the drug container 20A and the receiving space 15A. The more drug-releasing microfluidic channels 40A can release more medications at the same time. The purpose of the sustained release therapy for the dental caries in the receiving space 15A can be achieved by the present invention.

In summary, the sustained drug-releasing artificial dental crown can be capped on a tooth and then the sustained drug-releasing artificial dental crown can release the medications slowly and continuously to the tooth by the micro-structures of the drug container 20, 20A, the drug-supplying microfluidic channel 30, and the multiple drug-releasing microfluidic channels 40, 40A in the crown body 10, 10A. This arrangement enhances the effect of the local deposition and the sustained drug-releasing artificial dental crown helps to decrease the consumption of the medications, preventing the waste of the medications. In addition, the medications are only administrated by local deposition on the surface of the tooth. The medications are not delivered to the other organs in patients' body. The present invention is suitable for not only pregnant and hepatorenal syndrome patients but also for patients who can't take care of the dental hygiene or who can't take the medications by themselves.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. A sustained drug-releasing artificial dental crown comprising:
a crown body comprising
an upper portion,
a bottom portion,
a sidewall connected to the upper portion, and
a cavity encompassed by the sidewall below the upper portion;
a drug container defined inside the upper portion of the crown body;
a drug-supplying microfluidic channel connecting the drug container and an exterior of the crown body; and
a drug-releasing microfluidic channel connecting the drug container and the cavity.

2. The dental crown as claimed in claim 1, wherein the drug container is extended inside the sidewall from the upper portion of the crown body.

3. The dental crown as claimed in claim 1, wherein the dental crown has multiple drug-releasing microfluidic channels.

4. The dental crown as claimed in claim 2, wherein the dental crown has multiple drug-releasing microfluidic channels.

5. The dental crown as claimed in claim 1, wherein the sidewall is gradually thinner from the upper portion towards the bottom portion.

6. The dental crown as claimed in claim 2, wherein the sidewall is gradually thinner from the upper portion towards the bottom portion.

7. The dental crown as claimed in claim 3, wherein the sidewall is gradually thinner from the upper portion towards the bottom portion.

8. The dental crown as claimed in claim 4, wherein the sidewall is gradually thinner from the upper portion towards the bottom portion.
